(19) United States
(12) Patent Application Publication
(10) Pub. No.: US 2003/0219867 A1
(43) Pub. Date: Nov. 27, 2003

(54) Clavulanic acid dehydrogenase, preparation and use for the production of clavulanic acid

(75) Inventors: Janice Arnell, Worthing (GB); Stephen William Elson, Madrid (ES); Neville Hubert Nicholson, Suffolk (GB); Stefan Roland Woroniecki, Horsham (GB)

Correspondence Address: GLAXOSMITHKLINE, Corporate Intellectual Property - UW2220, P.O. Box 1539, King of Prussia, PA, 19406-0939, US

(73) Assignee: SmithKline Beecham plc

(21) Appl. No.: 10/367,221

(22) Filed: Feb. 14, 2003

Related U.S. Application Data
(63) Continuation-in-part of application No. 08/586,664, filed on Apr. 3, 1996.
Application No. 08/586,664 is a 371 of international application No. PCT/EP94/02346, filed on Jul. 15, 1994.

(30) Foreign Application Priority Data
Jul. 24, 1993 (GB) 9315393.0

(51) Int. Cl. C12P 17/16; C12N 9/06; C07H 21/04; C12N 1/21; C12P 21/02; C12N 15/74

(52) U.S. Cl. 435/69.1; 435/191; 435/252.3; 435/320.1; 536/23.2; 435/118

(57) ABSTRACT

An isolated DNA fragment encoding a protein with CAD activity wherein the protein is clavulanic acid dehydrogenase (CAD) from S. clavuligerus, with the proviso that the isolated DNA fragment is not: (a) the 13.1 kb BglII to SphI fragment of S. clavuligerus; (b) the 10.8 kb EcoRI to BamHI fragment of S. clavuligerus; (c) the 8.8 kb BclI to BclII fragment of S. clavuligerus; (d) the 2.5 kb BglII to BglII fragment of S. clavuligerus; (e) the 60 kb BglII to BamHI fragment of S. clavuligerus; or (f) the 18.0 BclI to SphI fragment of S. clavuligerus.

### CROSS REFERENCE TO RELATED APPLICATIONS

This application is a continuation-in-part of U.S. Ser. No. 08/586,664 filed on Apr. 3, 1996 which is hereby incorporated by reference in its entirety and which is a 371 of International Application No. PCT/EP94/02346 filed Jul. 15, 1994, which claims benefit from foreign application GB 9315393.0 filed Jul. 24, 1993.

### FIELD OF THE INVENTION

The present invention relates to processes for preparation of an intermediate involved in the synthesis of clavulanic acid and an enzyme useful in the synthesis of clavulanic acid from intermediates involved in its biosynthesis. The present invention further relates to DNA molecules carrying a gene coding for an enzyme involved in clavulanic acid biosynthesis.

### BACKGROUND

The biosynthetic route to the important product clavulanic acid, a key ingredient of the antibiotic AUGMENTIN (Trademark of SmithKline Beecham plc) has been the subject of considerable study with a view to increasing the yield (titre) of clavulanic acid in fermentation processes. Clavulanic acid has been shown to be derived biosynthetically from clavaminic acid.

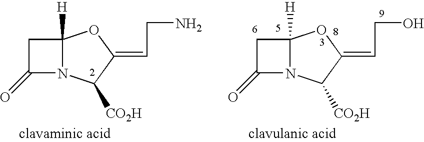

It has been reported (J. Chem. Soc., Chem. Commun. 1987,1737 and 1990, 617) that clavaminic acid is formed by the action of an enzyme on a precursor known as proclavaminic acid (see also EP-A-0213914). Earlier steps in the biosynthetic pathway have been more difficult to elucidate.

The final reaction in the biosynthesis of clavulanic acid involves the reduction of a side chain aldehyde to the hydroxyl group, using NADPH as the hydrogen donor.

The involvement of an aldehyde in the biosynthetic pathway has been suggested previously (Townsend et al. 1988, J Chem Soc Chem Commun, 18:1234-36; Iwata-Reuyl, D and Townsend, C. A. 1992, J AM Chem Soc 114:27624). Furthermore, the compound of formula (I):

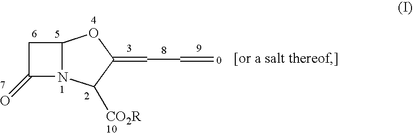

in which R is hydrogen or sodium salt thereof was identified in the parent application U.S. Ser. No. 08/586,664.

Compound (I) (“clavulanic acid aldehyde”) is approximately 100 times more active as a β-lactamase inhibitor than clavulanic acid. Compound (I) has a half life of about 0.5 h in aqueous solution at 20° C. therefore it is important that it is reduced to clavulanic acid as fast as possible during biosynthesis in order to maximise the titre of clavulanic acid.

Jensen (1992) referred to a partially sequenced 11 kb fragment of DNA from S. clavuligerus at the 5th conference of the American Society of Microbiology (October 11-15, Abstract A27) and suggested that this fragment contained the complete gene complement required for the biosynthesis of a β-lactamase inhibitor assumed to be clavulanic acid. However, the functions of the putative genes were not given and corresponding enzymes were not identified or isolated.

An enzyme having clavulanic acid dehydrogenase (hereinafter CAD) activity is capable of converting compound (1) into clavulanic acid under suitable conditions, in particular where compound (I) has the 2R, 5R stereochemistry. The enzyme having CAD activity is obtainable from microorganisms especially, Streptomyces species, preferably Streptomyces clavuligerus (e.g., ATCC 27064), S. jumonjensis (e.g., ATCC 29864) or S. katsurahamanus (e.g., T272).

An enzyme having CAD activity can be prepared by treating S. clavuligerus mycelium by centrifugation and ultrasonication followed by fractionation by ion-exchange chromatography. The enzyme is preferably in purified form, advantageously substantially pure form.

### SUMMARY OF THE INVENTION

The enzyme displaying CAD activity as discussed herein has an apparent molecular mass of 28 kD (by SDS PAGE). It also includes the sequence of N-terminal amino acids:

SEQ ID NO:3 PSALQGKVALITGASSGIGE

In particular, the enzyme comprises the sequence of amino acids in FIG. 1 or SEQ ID NO:1.

DNA encoding a protein with CAD activity is represented by DNA that comprises the DNA sequence in FIG. 1 or SEQ. ID. NO. 2.

The gene encoding said protein is located within the DNA fragment (F-I) shown in FIG. 2. Fragment (F-IV) as shown in FIG. 2 is a preferred subfragment according to the invention and which contains the whole CAD gene.

Also presented is a vector comprising such DNA, preferably an expression vector for expressing CAD in a suitable host organism. A specific example of such an expression vector is pIJ702 described by Katz., E., Thompson, C. J., Hopwood, D. A. (1983) Journal General Microbiology, 129:2703-2714. A preferred vector is pWOR14 (FIG. 3) which is DNA fragment (F-IV) ligated to pU702.

The current invention provides an isolated DNA fragment encoding a protein having CAD activity that hybridizes with an isolated 4.9 kb DNA encoding a protein with CAD activity and having the endonuclease restriction pattern shown in fragment F-(IV) in FIG. 2 wherein the protein encoding sequence in the region bounded by EcoRI and BglII restriction sites is as shown in SEQ ID NO: 2, with the proviso that the isolated DNA fragment is not: (a) the 13.1 kb BglII to SphI fragment of S. clavuligerus; (b) the 10.8 kb EcoRI to BamHI fragment of S. clavuligerus; (c) the 8.8 kb BclI to BclI fragment of S. clavuligerus; (d) the 2.5 kb BglII to BglII fragment of S. clavuligerus; (e) the 60 kb BglII to BamHI fragment of S. clavuligerus; or (f) the 18.0 kb BclI to SphI fragment of S. clavuligerus. In another aspect of the invention, a vector comprising the DNA is provided. In another aspect of the invention, a microorganism transformed with the DNA is provided. In yet another aspect of the invention, the microorganism contains multiple copies of the DNA. As used herein “multiple copies” of DNA refers to more than one repeat of an isolated DNA sequence.

In another embodiment of the invention, an isolated DNA fragment encoding a protein with CAD activity wherein the DNA fragment is obtainable from S. clavuligerus and comprises the sequence in SEQ ID NO: 2, with the proviso that the isolated DNA fragment is not: (a) the 130.1 kb BglII to SphI fragment of S. clavuligerus; (b) the 10.8 kb EcoRI to BamHI fragment of S. clavuligerus; (c) the 8.8 kb BclI to BclI fragment of S. clavuligerus; (d) the 2.5 kb BglII fragment of S. clavuligerus; (e) the 60 kb BglII to BamHI fragment of S. clavuligerus; or (f) the 18.0 kb BclI to SphI fragment of S. clavuligerus is provided. In another aspect of the invention, a vector comprising the DNA is provided. In another aspect of the invention, a microorganism transformed with this DNA is recited. In yet another aspect of the invention, the microorganism contains multiple copies of the DNA.

In yet another embodiment of the invention, an isolated DNA fragment encoding a protein with CAD activity wherein the protein is CAD from S. clavuligerus having an apparent molecular mass of 28 kD as determined by SDS PAGE and comprising the sequence of amino acids beginning at the N-terminus as shown in SEQ ID NO:1, with the proviso that the isolated DNA fragment is not: (a) the 130.1 kb BglII to SphI fragment of S. clavuligerus; (b) the 10.8 kb EcoRI to BamHI fragment of S. clavuligerus; (c) the 8.8 kb BclI to BclI fragment of S. clavuligerus; (d) the 2.5 kb BglII to BglII fragment of S. clavuligerus; (e) the 60 kb BglII to BamHI fragment of S. clavuligerus; or (f) the 18.0 BclI to SphI fragment of S. clavuligerus. In another aspect of the invention, a vector comprising the DNA is provided. In another aspect of the invention, a microorganism transformed with this DNA is recited. In yet another aspect of the invention, the microorganism contains multiple copies of the DNA.

### BRIEF DESCRIPTION OF THE FIGURES

FIG. 1 is a nucleic acid sequence and encoded amino acid sequence of a protein with CAD activity. This nucleic acid sequence is presented as SEQ ID NO:2 and the protein sequence is presented as SEQ ID NO:1.

FIG. 2 is a restriction map of part of the clavulanic acid gene cluster. Within FIG. 2, F-(1) is the 130.1 kb BglII to SphI fragment of S. clavuligerus; F-(II) is the 10.8 kb EcoRI to BamHI fragment of S. clavuligerus; F-(III) is the 2.5 kb BglII to BglII fragment of S. clavuligerus; and F-(IV) is the 4.9 kb SphI to SphI fragment of S. clavuligerus.

FIG. 3 is the vector, pWOR14, showing some restriction site.

FIG. 4 shows β-lactamase inhibition against time for clavulanic acid and dcl I 111 broth supernatant.

FIG. 5 shows three restriction fragments of S. clavuligerus DNA. Within FIG. 5, F-(V) is the 60 kb BglII to BamHI fragment of S. clavuligerus, F-(VI) is the 18.0 kb BclI to SphI fragment of S. clavuligerus, and F-(VII) is the 8.8 kb BclI to BclI fragment of S. clavuligerus.

### DETAILED DESCRIPTION OF THE INVENTION

Definitions

“Isolated” means altered “by the hand of man” from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated,” as the term is employed herein. “Polynucleotide” generally refers to any polyribonucleotide or polydeoxribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for instance, PROTEINS—STRUCTURE AND MOLECULAR PROPERTIES, 2nd Ed., T. E. Creighton, W. H. Freeman and Company, New York, 1993 and Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York, 1983; Seifter, et al., “Analysis for protein modifications and nonprotein cofactors”, Meth Enzymol (1990) 182:626-646 and Rattan, et al., “Protein Synthesis: Posttranslational Modifications and Aging”, Ann NY Acad Sci (1992) 663:48-62.

“Variant” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

“Identity” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SLAM J. Applied Math., 48: 1073 (1988)). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F., et al., J. Molec. Biol. 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215: 403410 (1990). The well known Smith Waterman algorithm may also be used to determine identity.

Preferred parameters for polypeptide sequence comparison include the following:

1) Algorithm: Needleman and Wunsch, J. Mol Biol. 48: 443453 (1970)

Comparison matrix: BLOSSUM62 from Hentikoff and Hentikoff, Proc. Natl. Acad. Sci. USA. 89:10915-10919 (1992)

Gap Penalty: 12

Gap Length Penalty: 4

A program useful with these parameters is publicly available as the “gap” program from Genetics Computer Group, Madison Wis. The aforementioned parameters are the default parameters for peptide comparisons (along with no penalty for end gaps).

Preferred parameters for polynucleotide comparison include the following:

1) Algorithm: Needleman and Wunsch, J. Mol Biol. 48: 443-453 (1970)

Comparison matrix: matches=+10, mismatch=0

Gap Penalty: 50

Gap Length Penalty: 3

Available as: The “gap” program from Genetics Computer Group, Madison Wis.

These are the default parameters for nucleic acid comparisons.

By way of example, a polynucleotide sequence of the present invention may be identical to the reference sequence of SEQ ID NO:2, that is it may be 100% identical, or it may include up to a certain integer number of amino acid alterations as compared to the reference sequence such that the percent identity is less than 100% identity. Such alterations are selected from the group consisting of at least one nucleic acid deletion, substitution, including transition and transversion, or insertion, and wherein said alterations may occur at the 5′ or 3′ terminal positions of the reference polynucleotide sequence or anywhere between those terminal positions, interspersed either individually among the nucleic acids in the reference sequence or in one or more contiguous groups within the reference sequence. The number of nucleic acid alterations for a given percent identity is determined by multiplying the total number of amino acids in SEQ ID NO:2 by the integer defining the percent identity divided by 100 and then subtracting that product from said total number of amino acids in SEQ ID NO:2, or:

nₙ=xₙ−(xₙy),

wherein nₙ is the number of amino acid alterations, xₙ is the total number of amino acids in SEQ ID NO:2, y is, for instance 0.70 for 70%, 0.80 for 80%, 0.85 for 85% etc., is the symbol for the multiplication operator, and wherein any non-integer product of xₙ and y is rounded down to the nearest integer prior to subtracting it from xₙ.

Preferred polypeptide embodiments further include an isolated polypeptide comprising a polypeptide having at least a 50,60, 70, 80, 85, 90, 95, 97 or 100% identity to a polypeptide reference sequence of SEQ ID NO:1, wherein said polypeptide sequence may be identical to the reference sequence of SEQ ID NO:1 or may include up to a certain integer number of amino acid alterations as compared to the reference sequence, wherein said alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence, and wherein said number of amino acid alterations is determined by multiplying the total number of amino acids in SEQ ID NO:1 by the integer defining the percent identity divided by 100 and then subtracting that product from said total number of amino acids in SEQ ID NO:1, or:

nₐₓₐ−(xₐy),

wherein nₐ is the number of amino acid alterations, xₐ is the total number of amino acids in SEQ ID NO:2, y is 0.50 for 50%, 0.60 for 60%, 0.70 for 70%, 0.80 for 80%, 0.85 for 85%, 0.90 for 90%, 0.95 for 95%, 0.97 for 97% or 1.00 for 100%, and is the symbol for the multiplication operator, and wherein any non-integer product of xₐ and y is rounded down to the nearest integer prior to subtracting it from xₐ.

By way of example, a polypeptide sequence of the present invention may be identical to the reference sequence of SEQ ID NO:1, that is it may be 100% identical, or it may include up to a certain integer number of amino acid alterations as compared to the reference sequence such that the percent identity is less than 100% identity. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in SEQ ID NO:2 by the integer defining the percent identity divided by 100 and then subtracting that product from said total number of amino acids in SEQ ID NO:1, or:

nₐ=xₐ−(xₐy),

wherein nₐ is the number of amino acid alterations, xₐ is the total number of amino acids in SEQ ID NO:2, y is, for instance 0.70 for 70%, 0.80 for 80%, 0.85 for 85%, etc., and is the symbol for the multiplication operator, and wherein any non-integer product of xₐ and y is rounded down to the nearest integer prior to subtracting it from xₐ.

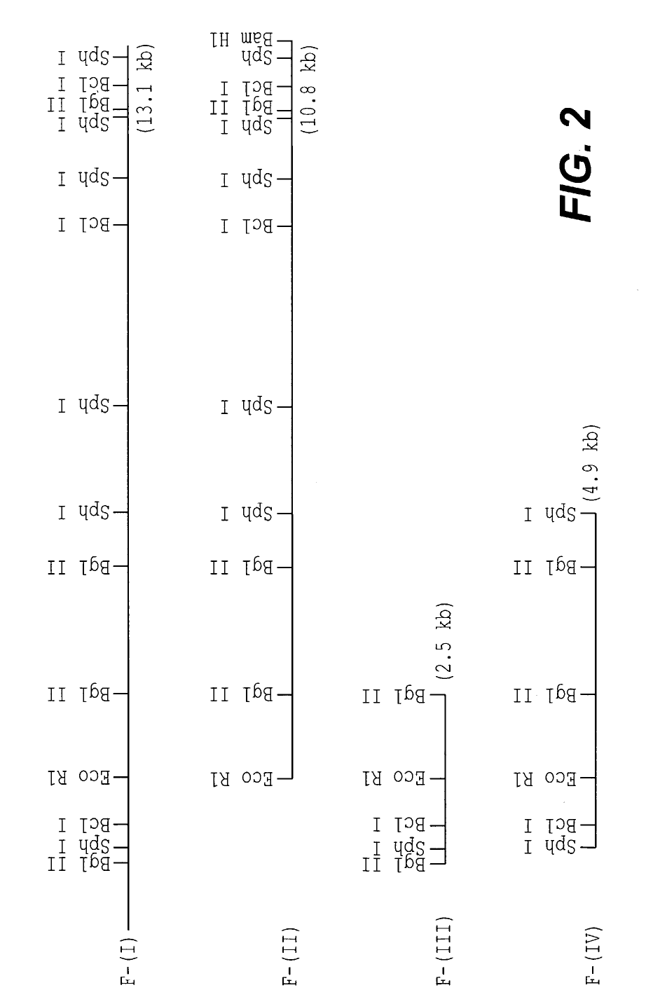

In FIGS. 2, 3 and 5 the abbreviations EcoRI, BgIII, etc. are conventional abbreviations for enzyme restriction endonucleases and the approximate length in kilobases (kb) of the DNA as determined by sizing experiments carried out by agarose gel electrophoresis, is indicated. It should be understood that the Figures are not intended to show all the enzyme restriction sites present on the DNA fragments indicated.

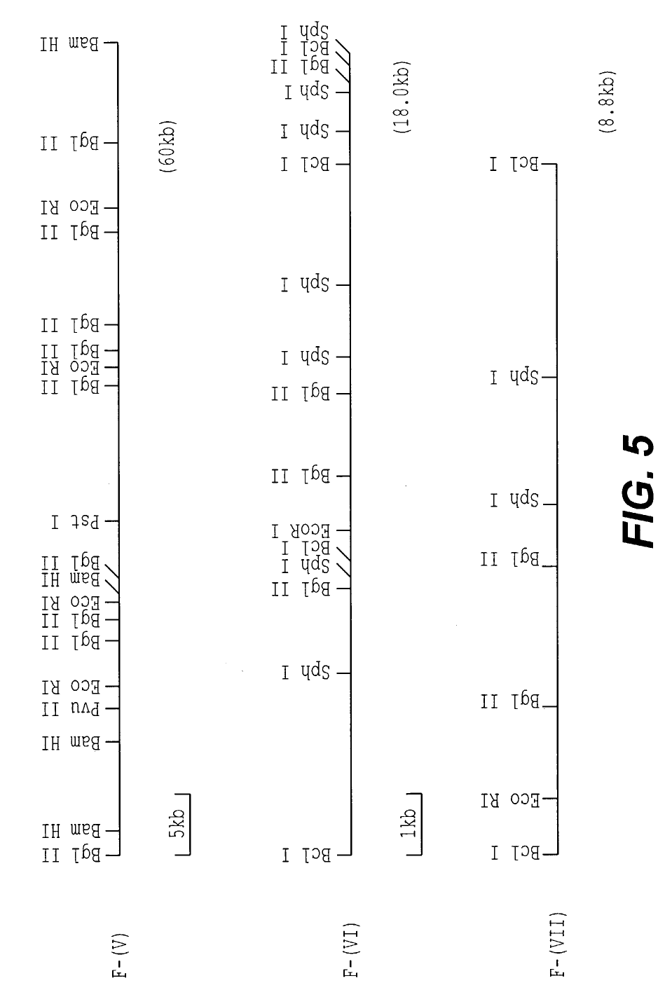

FIG. 5 describes three restriction fragments of S. clavuligerus DNA. Within FIG. 5, F-(V) is the 60 kb BglII to BamHI fragment of S. clavuligerus, F-(VI) is the 18.0 kb BclI to SphI fragment of S. clavuligerus, and F-(VII) is the 8.8 kb BclI to BclII fragment of S. clavuligerus.

It will be understood that the DNA of this invention is not in its natural state as it occurs in nature but is in isolated or substantially pure form.

It will be understood that the invention encompasses DNA which may not have the precise configuration of restriction sites illustrated if the said DNA has been derived by standard techniques including nucleotide deletion, substitution, addition or inversion from the DNA according to any aspect of the invention described above.

Preferably the DNA of the present invention is derived from S. clavuligerus ATCC 27064. However the invention also encompasses DNA sequences derived from other suitable organisms especially clavulanic acid producing organisms other than S. clavuligerus, which sequences do not have the configuration of restriction sites shown in FIGS. 2, 3 or 5 but which hybridize, preferably under conditions of high stringency, with the DNA shown in FIG. 2, 3 or 5 a subfragment thereof and which code for CAD or an enzyme with CAD activity.

As herein used, the terms “stringent conditions” and a “stringent hybridization conditions” mean hybridization will occur only if there is at least 70% and preferably at least 80%, but especially preferably at least 95% identity between the sequences. An example of stringent hybridization conditions is overnight incubation at 42° C. in a solution comprising: 50% formamide, 5×SSC (150 mM NaCl, 15 mM trisodium citrate), 50 mM sodium phosphate (pH 7.6), 5× Denhardt's solution, 10% dextran sulfate, and 20 micrograms/ml denatured, sheared salmon sperm DNA, followed by washing the filters in 0.1×SSC at about 65° C. Hybridization and wash conditions are well known and exemplified in Sambrook, et al., Molecular Cloning: A Laboratory Manual, Second Edition, Cold Spring Harbor, N.Y., (1989), particularly Chapter 11 therein, the disclosure of which is hereby incorporated in its entirety by reference.

As used herein an enzyme with “CAD activity” refers to an enzyme that is capable of converting compound (I) into clavulanic acid under suitable conditions, in particular where compound (I) has the 2R, 5R stereochemistry. CAD activity can be measured as one non-limiting example, by exposing microbial culture to clavulanic acid aldehyde and then measuring clavulanic acid production of the microorganism using a standard zone of inhibition. CAD activity can be correlated to the diameter of the zone observed, a technique that is well known in the art.

Similarly, an aqueous solution of compound (I) (0.11 ml, 10 mM) can be added to washed S. clavuligerus SC2 cells (0.5 ml) which are sonicated after 48 hours growth. The suspension is then treated with 10 mM NADPH (0.11 ml), made up to 1 ml with 50 mM MOPS buffer at pH 7.0 and incubated in stoppered tubes at 26° C. Samples (0.2 ml) can be treated with methanol (0.2 ml) for 5 minutes and centrifuged. The clear supernatants are made up to 1.6 ml with MOPS buffer and assayed on discs by the KaG method, which is well known in the art. Undiluted reaction mixture is applied to Whatman No. 1 chromatography tapes which can be developed in either butan-1-ol-acetic acid-water (12:3:5) or butan-1-ol-ethanol-water (7:1:2), dried, and visualized by application to a KaG bioassay plate. The presence of clavulanic acid in the supernatant is indicative of CAD enzyme activity. (Fulston et al., J. Chem. Soc., Perkin Trans 1, 2001, 1122-1130.)

The DNA of the invention and vectors containing the same may find use in many areas of industrial activity. That also applies to host microorganisms transformed with said vectors and the enzymes they express. For example, the DNA may be utilised as a hybridization probe to identify and isolate related or overlapping genes present on the total cellular DNA of S. clavuligerus (ATCC 27064) and of other microorganisms which produce enzymes of similar structure and specificity. Recombinant vectors containing said DNA may be of value, when transformed into suitable hosts, in the production of genetically modified microorganisms that synthesize increased amounts of clavulanic acid. For example, as demonstrated herein, as clavulanic acid aldehyde is relatively shortlived in vitro/in vivo, it would be very advantageous to increase the amount of activity of CAD in a suitable organism. Recombinant vectors could also be used in the generation of novel or hybrid antibiotics via the process of gene transfer (see for example D. A. Hopwood et al., (1985) Nature 314:642-644). Enzymes encoded by the DNA of the invention may be used, for example, in cell-free systems especially when immobilised on suitable solid supports, to prepare the known antibiotic from natural precursors or a novel antibiotic from ‘unnatural’ precursors obtained, for example, by chemical synthesis.

The DNA of the invention or a fragment thereof (not necessarily carrying an intact gene) may be combined, either by recombinant DNA techniques or by natural recombination processes, with a fragment of a gene involved in biosynthesis to produce a hybrid gene capable of directing the synthesis of a hybrid enzyme. Such enzymes may be used in the production of novel antibiotics by processes analogous to those hereinbefore described.

The DNA of the invention may also be modified by the known techniques of site-directed mutagenesis (in a manner analogous to that described, for example, by G. Winter et al, (1982) Nature 299:756-758; or by Zoller and Smith, (1982) Nucleic Acids Research 10:6487-6500) to give DNA in which specific mutations and/or deletions have been effected. The mutated DNA may be used to obtain an increased yield (or titre) of clavulanic acid from a suitable host microorganism. The mutated DNA may also be used to obtain novel or hybrid antibiotics by gene transfer, or used in the production of mutant enzymes (muteins) which may be used in the production of novel antibiotics by analogous processes to those hereinabove described.

The following examples illustrate the invention. These examples do not limit the scope of this invention which is defined by the appended claims.

### EXAMPLE 1

Clavulanic Acid Biosynthesis: Involvement of an Aldehyde Intermediate

The aldehyde 3-oxoethylidene-7-oxo-4-oxa-1-azabicyclo-[3.2.0]heptane-2-carboxylic acid i.e. compound (I) has been chemically synthesized and shown to possess β-lactamase inhibitory activity. The aldehyde can also be prepared by fermentation.

Compound (I) decomposed spontaneously in aqueous solution with a half life of one hour, but could be reduced to clavulanic acid by an NADPH dependent enzyme present in Streptomyces clavuligerus SC2. A chloroform solution of the aldehyde benzyl ester (11) was found to invert its stereochemistry spontaneously at C3 and C5 at room temperature; and this result has provided a hypothetical mechanism for the inversion of stereochemistry found to occur during clavulanic acid biosynthesis. It is concluded that compound (I) occurs as a late stage intermediate in clavulanic acid biosynthesis. This pathway must include an inversion of the stereochemistry of the 3 and 5 positions of clavaminic acid. The following experiments provide evidence for the involvement of the aldehyde (I) in clavulanic acid biosynthesis.

Discussion

The aldehyde (Compound (1) was prepared as a mixture of geometric isomers about the double bond between C3 and C8 E:Z (2:1) by hydrogenolysis of the benzyl ester (II) in a yield which was estimated to be 35% by titration with sodium hydroxide solution. An h.p.l.c. assay for (I) was developed with ultra violet detection of the allylic aldehyde chromophore at 260 nm. Fractions of the h.p.l.c. eluent which contained the chromophore were evaporated to dryness and shown to contain (1) by NMR spectroscopy. Repeated h.p.l.c. analysis of a solution of the salt (I) showed that, at room temperature, about half had decomposed in one hour. This instability prevented full characterization of the acid (1), so it was converted with diazomethane to its methyl ester (IV) in an overall yield of 16% from the benzyl ester (II) (A. G. Brown et al, J. Chem. Soc. Perkin Trans. 1, 1984, 635). The spectral data obtained for the methyl ester (IV) were indistinguishable from those obtained by A. G. Brown et al., 1984.

Compound (I) was reduced to clavulanic acid by NADPH in the presence of a broken cell suspension of S. clavuligerus SC2 (Table 1). (SC2 is a reisolate of ATCC 27064). The clavulanic acid produced was detected by KaG bioassay (AG. Brown, et al. (1976) J. Antibiot. 29:668) and chromatographic tapes loaded with the reaction mixture, were compared with standards to confirm the presence of clavulanic acid. The reduction was complete before a sample could be removed from the reaction vessel, but appeared to give clavulanic acid in only 14% yield. However, should the enzyme be capable of reducing only the Z isomer of (I) (the geometry of clavulanic acid), the yield is 42%. The failure of the reduction when NADH was used in place of NADPH and when the broken cell suspension was absent or boiled, indicates that the reaction is catalyzed by an NADPH dependent enzyme or enzymes present in the broken cell suspension.

TABLE 1
Reduction of (III) by a broken cell
suspension (BCS) of S. clavuligerus SC2

Clavulanic Acid (μg/ml) at time

| Reaction Mixture | 0 mins (KaG bioassay) | 60 mins |
|---|---|---|
| BCS' + (III) | 8.0 | 7.0 |
| BCS' + (III) + NADPH | 28.8 | 27.2 |
| BCS' + NADPH | 2.4 | 2.4 |
| (III) + NADPH | 8.8 | 7.2 |
| Boiled BCS' + (III) + | 6.6 | 6.6 |
| NADPH | | |
| BCS' + (III) + NADH | 6.2 | 6.6 |

'BCS is broken cell suspension (Woroniecki et al. (U.S. Pat. No. 4,795,809 ex 21) hereby incorporated by reference in its entirety). The concentration of NADH or NADPH above is 1 mM.

While the above demonstrates that S. clavuligerus can reduce (I) to clavulanic acid, it offers no explanation of the inversion of stereochemistry at the 2 and 5 positions during the biosynthesis of clavulanic acid. Certain authors have proposed theoretical schemes for this phenomenon [Recent Advances in the Chemistry of β-lactam antibiotics, G. I. Gregory, ed, 2nd International Symposium, Royal Soc. Chem (No 38) pl58]. However, we have found that when the aldehyde benzyl ester (II) in chloroform solution was examined by nm using enantioselective solvating reagent, and by optical rotation, it was found to racemise its stereochemistry at the two and five positions (scheme shown below) spontaneously at room temperature. An ester of the aldehyde might, therefore, be the biosynthetic intermediate at which the clavaminic acid stereochemistry is inverted to the clavulanic acid stereochemistry.

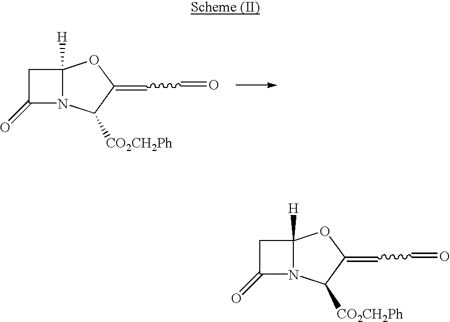

Experimental

Preparation of Sodium 3-oxoethylidene-7-oxo-4-oxa-1-azabicyclo (3.2.0)heptane-2-carboxylate (III)

Benzyl 3-oxoethylidene-7-oxo4-oxa-1-azabicyclo[3.2.0]heptane-2-c arboxylate (II) (50 mg, 0.17m mol) was dissolved in tetrahydrofuran (5 ml) and 0.2 M sodium phosphate adjusted to pH 6.85 (5 ml), and hydrogenated at ambient temperature and pressure for ten minutes with 10% palladium on carbon (40 mg). The reaction mixture was rapidly filtered and evaporated to dryness while the temperature was maintained below 10C. The residue was immediately redissolved in D₂O to give a solution of the title compound (III). δ_{H} (²⁵⁰ MHz, D₂O) inter alia E isomer 3.25 (1H, d, J 17.4 Hz, 6β-H), 3.65 (1H, dd, J 17.4 and 2.9 Hz, 6 a-H), 5.73 (1H, d, J 1.2 Hz, 2-H), 5.79 (1H, dd, J 8.3 and 1 Hz, 8-H), 5.94 (1H, d, 2.7 Hz, 5-H), 9.55 (1H, d, J 8.3 Hz, 9-H), Z isomer 3.29 (1H, d, J 17.3 Hz, 6β-H), 3.65 (1H, dd, J 17.4 and 2.9 Hz, 6 a-H), 5.27 (1H, s, 2-H), 5.50 (1H, d, J 8.2 Hz, 8-H), 6.00 (1H, d, J2.7 Hz, 5-H), and 9.70 (1H, d, J 8.2 Hz, 9-H). The E and Z isomers were assigned on the relative positions of their 8-proton resonance (Gerald Brooks+Eric Huit J. Chem. Soc. Perkin Trans. 1 (1983) 2513) and were in the ratio E-Z (2:1). Material prepared by the above hydrogenation with water substituting for the phosphate solution and adjustment of the pH of the reaction mixture to 6.5 with 0.1 M NaOH gave (III) (Yield 35% from NaOH consumed), λmax. (H₂O) 262 nm; νₘₐₓ (KBr) 3 400, 1 792 (br), and 1 640 cm⁻¹ (br), and 1 640 cm⁻¹ ¹ (br); thin layer chromatography on silica 60 eluted with butan-1-ol-ethanol-water (4:1:1) and visualized with Schiff's reagent gave a blue zone (Rf:0.52). Addition of a known weight of fumaric acid to the crude product solution followed by evaporation and nmr analysis indicated a yield of 20% by comparison of the intensity of the fumarate protons with the intensity of the aldehydic proton of (III). When this solution was allowed to stand at room temperature for 3.5 hours, (III) was seen to have decomposed to a yield of 3.2%.

μ-lactamase inhibition by (III): A solution of III was tested as a β-lactamase inhibitor by the automated chromogenic substrate assay of C. Reading and T. Farmer against Klebsiella E70 β-lactamase. Taking the yield of (III) from the hydrogenation reaction as 35% gave an 150 with pre-incubation of 0.01 μg/ml and without pre-incubation of enzyme and inhibitor of 1 μg/ml.

HPLC of (I)

Analytical: A Waters C₁₈ Microbondapak column was eluted at 2 mls/min with 0.1M sodium phosphate at pH 3.2. The eluent was monitored by an ultra violet detector at 260 nm. The retention time of (I) was 5.6 minutes.

Preparative: Half of the (III) prepared by hydrogenation was evaporated to a small volume, adjusted to pH 4.0 with 0.5M orthophosphoric acid and made up to 2 ml with water. This solution was chromatographed over a Waters C₁₈ Radpak cartridge supported in a Waters Z module and elected at 4 mls/min. with 0.1M sodium phosphate adjusted to pH 4.0. A fraction coincident with the elution of a strong chromophore at 260 nm was collected between 3.8 and 5.0 minutes. Evaporation of this fraction followed immediately by nmr gave a spectrum consistent with (III).

Spontaneous decomposition of (III):—A solution of (5) in 0.2 M aqueous sodium phosphate at pH 6.85 was allowed to stand at room temperature. H.p.l.c. analysis showed the solution to contain 85% of the original (III) after 35 minutes and 63% after 53 minutes.

Methyl 3-oxoethylidene-7-oxo-4-oxa-1-azabicyclo[3.2.0]heptane-2-carboxylate (IV):—Benzyl 3-oxoethylidene-7-oxo-4-oxa-1-azabicyclo[3.2.0]heptane-2-carboxylate (II) (100 mg, 0.35 ml) was hydrogenolysed as before to provide (I) in water (10 ml). This solution was acidified to pH 2.5 under a layer of ether (5 ml) at 4° C. and stirred for five minutes with each of three aliquots of diazomethane. After twenty minutes, excess diazomethane was removed by a stream of nitrogen and ethyl acetate (30 ml) was added. The organic layer was separated, dried with magnesium sulphate and chromatographed rapidly over silica gel in ethyl acetate-hexane (1:1)) to give the title compound as mixture of geometric isomers E-Z (1.8:1) (12 mg, 16% from the ester (II)). The spectral data obtained for these isomers were indistinguishable from those obtained from the previous preparation.

Enzymatic reduction of (III) to clavulanic acid:—solution of (III) (0.23 mg, 1 μmol, estimated by titration of acid (I)) in water (0.1 ml 1 μmol) was added to washed 48 hour S. clavuligerus SC2 cells which had been sonicated (EP 0213 914 example 20) (0.5 ml). The suspension was treated with 10 mM NADPH (0.1 ml, 1 μmol), made up to one millilitre with 50 mM MOPS buffer at pH 7.0 and incubated in stoppered tubes at 26° C. Samples (0.2 ml) were removed, treated with methanol (0.2 ml) for 5 minutes, and centrifuged. The clear supernatants were made up to 1.6 ml with MOPS buffer and assayed on discs by the KaG method. Undiluted reaction mixture was applied to Whatman No. 1 chromatography tapes which were developed in either butan-1-ol-acetic acid-water (12:3:5) or butan-1-01-ethanol-water (7:1:2), dried, and visualized by application to a KaG bioassay plate. Standard clavulanic acid solutions were developed and visualized similarly.

Racemisation of benzyl 3-oxoethylidene-7-oxo-4-oxa-1-azabicyclo[3.2.0] heptane-2-carboxylate (II):—A solution of the title compound (II) as a mixture of geometrical isomers E-Z (2:1) (162 mg, 0.56 mmol) in chloroform (8 ml) was allowed to stand at ambient temperature away from strong light for three days. The solution was evaporated to dryness, chromatographed over silica gel in ethyl acetate-hexane (1:1), and, on evaporation, gave the title compound in the ratio of geometric isomers E-Z (1.7:1) (53 mg, 33%), [α]²⁰+16.4° (c=2% in dichloromethane); addition of R-2,2,2-trifluoro-1-(9-anthryl)ethanol (1000% by weight) to the nmr solution (250 MHz, CDCl₃) caused two sets of signals to appear for the 6b, 6a and 8 protons of both the E and Z isomers and for the 2 proton of the E isomer. Irradiation of specific protons demonstrated coupling between the 5 and 6 and the 8 and 9 protons for all these signals. The intensity of the signals indicated a 65:35 mixture of enantiomers. When the title compound was freshly prepared the geometric isomer ratio was E-Z (1.6:1), [α]²⁰+53.9° (c=2% in dichloromethane); and addition of R-2,2,2 trifluro-1-(9-antreyl)ethanol to the nmr solution showed a 93:7 mixture of enantiomers.

Conclusion:

The presence of an enzyme in a clavulanic acid producing strain of S. clavuligerus which can reduce (I) to clavulanic acid suggests that (I) is a naturally occurring anabolite. The enantiomeric exchange demonstrated by the ester (II) can explain the inversion of stereochemistry which occurs during the biosynthesis of clavulanic acid from clavaminic acid but provides no proof that such a mechanism applies in vivo. Confirmation that compound (I) is a biosynthetic intermediate and that these mechanisms are operating, has been confirmed by the discovery of compound (I) naturally occurring in S. clavuligerus broth.

### EXAMPLE 2

Stereochemistry and Biosynthesis Experiments

Naturally occurring 3-oxoethylidene-7-oxo-4-oxa-1-azabicyclo[3.2.0]heptane-2-carboxylic acid (I) has been identified by h.p.l.c. at a concentration of circa 0.4 μg/ml in the broth of the clavulanic acid producing Streptomyces clavuligerus SC2 and circa 4 μg/ml in the broth of the clavulanic acid deficient mutant S. clavuligerus dc1 I 111. (The S. clavuligerus mutant Dcl I 111 was isolated during random screening procedures for high titre clavulanic acid producing mutants). A decarboxylated derivative (V) was isolated from the latter broth and shown to possess 5-R stereochemistry.

Fresh dcl I 111 broth gave strong β-lactamase inhibitory activity which decayed at the same rate as chemically synthesized 2R,5R-(1). The last two results indicate that the stereochemistry of naturally occurring (I) is 5R —the same as that of clavulanic acid; and it is therefore concluded that (I) is the immediate biosynthetic precursor to clavulanic acid.

Naturally occurring (I) was identified by h.p.l.c. in the clavulanic acid deficient mutant S. clavuligerus dcl I 111. These organisms were fermented for between 43 and 60 hours when their broth supernatants were extracted with methyl acetate at acidic pH. The extracts were concentrated and shown to possess an h.p.l.c. peak which had the same retention time and ultra violet absorption spectrum as (I) chemically synthesized from clavulanic acid. Quantitation of (I) is difficult due to its chemical instability, but approximation was made by comparison of the intensity of the broth derived h.p.l.c. peak either with that of synthetic (I), or with that of a more stable derivative possessing a similar chromophore. Comparison with synthetic (I) give a broth concentration for S. clavuligerus dcl I 111 of 4 μg/ml (I). Comparison with the h.p.l.c. peak of a solution of (V) of known concentration gave broth concentrations of (I) of half these values.

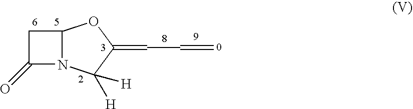

Isolation of (I) from I 111 broth is prevented by its chemical instability. However, synthetic (I) decarboxylates in good yield to give the derivative (V) with retention of stereochemistry at the 5 position. The derivative (V) is chemically and stereochemically stable in organic solution and survives rapid silica chromatography (recovery 70%). It is therefore suitable for isolation, characterization and stereochemical evaluation and this data will indirectly provide the corresponding data for naturally occurring (I). For these reasons (I) in 111 broth was converted by decarboxylation to the derivative (V).

The methyl acetate extract (MAE) of dc I 111 was allowed to stand at room temperature and the formation of the decarboxylated derivative (V) from natural (I) was monitored by h.p.l.c. The E isomer of (V) so formed was purified by chromatography over Sephadex LH 20 and high pressure semi-preparative silica chromatography. The nmr, ir, uv, h.p.l.c. and mass spectral data for this compound were indistinguishable from those for a sample of E-V chemically prepared from clavulanic acid. However, when enantiomeric solvating reagent (esr) was added to the nmr solution of the synthetic material, the doublet of doublets due to the 2β proton was split into two sets of signals in the ratio 2:1. When E-V from natural sources was treated in the same way, only one set of 2β proton signals could be observed. These results indicate that the synthetic material was a mixture of enantiomers at the 5 position, while the naturally derived E-V was a single enantiomer. The enantiomeric purity of the natural material demonstrates that only a single isomer of (I) exists in vivo and that the S position of this molecule does not undergo racemisation either in (I) itself or when that molecule decarboxylates. The synthetic E-V was a mixture of enantiomers due to the previously reported racemisation of the benzyl ester (II) from which it was made; and, since the ester (II) was prepared from 2R, 5R clavulanic acid, the major isomer in synthetic E-V must be 5R. In order to determine the absolute stereochemistry of the naturally derived E-V, the solution of this material which had been treated with esr was added to the similarly treated solution of synthetic E-V. NMR spectroscopy showed that the intensity of the major set of 6β proton signals had increased relative to the minor. Naturally occurring (I) must, therefore, possess the SR stereochemistry.

Circular dichroism spectroscopy provides corroboration for these conclusions. The decarboxylated products E-V share the same chromophore and a positive Δε, but the intensity of the absorption of the natural material is 2.4 times that of the synthetic material. Thus, natural E-V has the SR stereochemistry and must be derived from (I) with SR stereochemistry.

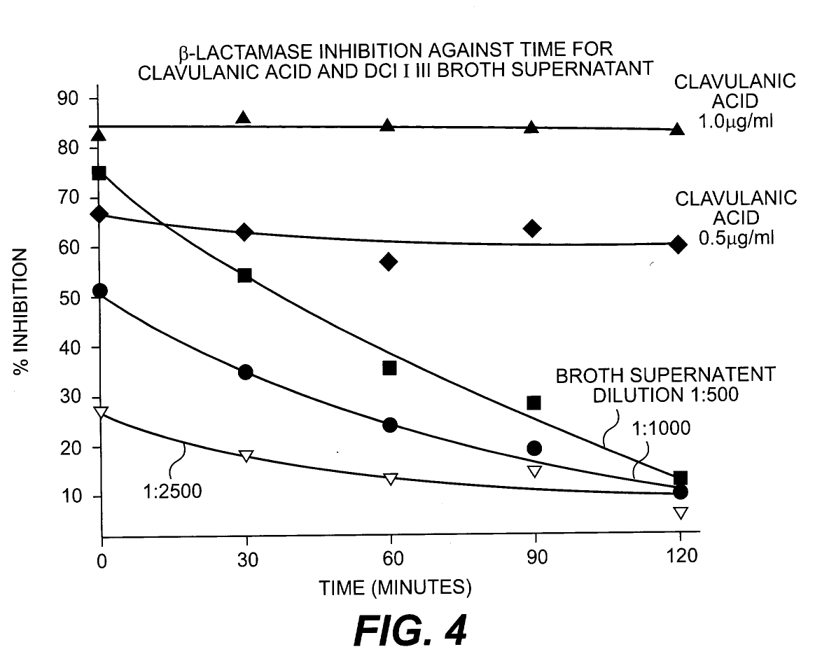

Both the presence and stereochemistry of (I) in dcl I 111 may be inferred from the β-lactamase inhibitory activity of the broth supernatant. Fresh samples of 45 hours I 111 supernatant were tested by the chromogenic cephalosporin assay Antibiotic: Assessment of antimicrobial activity and resistance (A. Denver Russell and Louis B (eds). Quesxel—page 147.) and shown to possess β-lactamase inhibitory activity against R TEM JT4 equivalent to that of a solution of 375 mg/ml of clavulanic acid. Unlike the clavulanic acid solution, the activity of the broth supernatant decayed with an approximate half life of one hour at room temperature (FIG. 4). Strong β-lactamase activity and the same rate of decomposition were found in (I) chemically synthesized from 2R, 5R clavulanic acid, so similar transient β-lactamase inhibitory activity in I 111 broth may be attributed to the presence of (I). Previous publications (S. W. Elson, J. Gillett, N. H. Nicholson, and J. W. Tyler, J. Chem. Soc. Chem. Commun. 1988 979; S. W. Elson, K. H. Baggaley, J. Gillett, S. Holland, N. H. Nicholson, J. T. Sime, and S R. Woroniecki, J. Chem. Soc. Chem Commun. (1987) 1937) have shown that, although clavulanates with the 2R, 5R stereochemistry possess strong β-lactamase inhibitory activity, those possessing the 2S,5S stereochemistry do not. This activity therefore, is consistent with 2R, SR stereochemistry for the (I) in dcl I 111 broth.

In summary, the presence of (I) in S. clavuligerus broth has been demonstrated by h.p.l.c. and ultra-violet spectroscopic comparison with a synthetic standard, by its β-lactamase inhibitory activity and by isolation and characterization of a decarboxylated derivative. The stereochemistry of natural (I) was confirmed as 5-R by its β-lactamase inhibitory activity and by comparison of the decarboxylated derivative with the same compound of known stereochemistry.

Conclusion:

SC2 has been shown to possess an NADPH dependent enzyme capable of reducing (1) to clavulanic acid. Here, we have shown that the clavulanic acid deficient mutant dcl I 111 accumulates (I) to a steady state concentration, determined by its rate of production and decomposition, which is ten fold higher that the concentration found in SC2. From these results we deduce that I 111 is unable to produce clavulanic acid because of a deficiency in the enzyme performing the reduction and that (I) is, therefore, on the biosynthetic pathway to clavulanic acid. Since (I) and clavulanic acid share the 5-R stereochemistry we conclude that (I) is the immediate biosynthetic precursor to clavulanic acid.

Extraction of (I) from S. clavuligerus Broth:

S. clavuligerus dcl I 111 was fermented essentially as in European patent 0213 914 example 20. At 50 hours, the broth was rapidly cooled to 2° C. The methyl acetate extract (MAE) was maintained at less than 10° C. and was used immediately in the following experiments.

H.p.l.c. assay for (I) in S. clavuligerus broth:—A sample of the methyl acetate extract (MAE) (10 ml) was evaporated to an aqueous residue, readjusted to pH 3.2 and made up to a known volume of about 0.5 ml. This sample was immediately compared with a fresh, chemically prepared sample of (I) by h.p.l.c. (conditions analytical) and gave a peak equivalent to 4 μg/ml of (I) for the I 111 culture broth. A similar assay using broth S. clavuligerus SC2 gave a broth concentration of 0.4 μg/ml (I).

Hplc Conditions B (for derivative (V)): Waters Microporasil Radpak column supported by RCM 8×10 and eluted at 2 mls/min with chloroform-hexane-ethanol (3:6:0.1). Ultra-violet detection at 255 nm using a diode array monitor. V—retention time 6.0 minutes; λmax 255 nm; 2.5%g gives an absorption of 0.25 OD.

C (for Derivative (V)): As B Eluting with Dichloromethane Hexane-Ethanol (3:6:0.1).

Purification of 5R-E-3-oxoethylidene-7-oxo-4-oxa-1-azabicyclo-[3.2.0]heptane (E-V) from S. clavuligerus dc1 I111 broth:—The methyl acetate extract (MAE) (1.15 liters) was allowed to stand at room temperature for four hours when h.p.l.c. (conditions B) showed no further increase in the peaks due to the decarboxylated product The solution was then evaporated to a small volume, the residue diluted with chloroform (200 ml) and the resulting aqueous phase (50 ml) discarded. The organic phase was washed with 1M sodium phosphate solution at pH 6.5, dried with magnesium sulphate and evaporated to dryness. This residue was chromatographed over Sephadex LH 20 in ethyl acetate-hexane (1:1) to give an oil (40 mg oil containing 2.0 mg (E-V); purity 5.0%). Material prepared in this fashion (30 mg) was chromatographed repeatedly (conditions B followed by C) and gave 5R-E-3-oxoethylidene-7-oxo-4-oxa-1-azabicyclo[3.2.0]heptan e (0.8 mg); CDΔ E_{237.4}14.6 (CH₃CN); (Found M⁺ 153.0438. Calc. for C₇H₇NO₃: M⁺ 153.0426); λₘₐₓ. (CH₃CN) 249 nm (ε14, 187 dm³ mol⁻¹ cm⁻¹); Vₘₐₓ. (KBr) 1 797, 1 668, 1 637, and 1 592 cm⁻¹; δ_{H} (250 MHz, CDCl₃) 3.12 1H, d, J 16.7 Hz, 6β-H), 3.54 (1H, ddd, J 0.9, 2.8 and 16.6 Hz, 6α-H), 3.92 (1H, d, J17.5 Hz, 2α-H), 5.01 (1H, dd, J1.6 and 17.5 Hz, 2β-H), 5.66 (1H, d, J2.7 Hz, 5-H), 5.82 (1H, ddd, J 1.7, 1.7 and 3.6 Hz, 8-H) and 9.50 (1H, J 4.9H₃, 9-H). Irradiation of the 8-H signal caused a collapse of coupling at the 9-H signal, loss of the smaller coupling at the 2β-H and sharpening of the 2α-H signal. Irradiation of the 2α-H signal caused loss of the smallest coupling at 6α-H. Addition of R-2,2,2-trifluoro-1-(9-anthyl)ethanol (1000%) caused no bifurcation of the signal due to the 2 β proton. Addition of the latter nmr solution to a solution demonstrating a 2:1 (R:S) mixture of enantiomers, showed a relative reduction in the intensity of the 2β proton of the 5S-enantiomer m/z 153 (M⁺, 9%), 136 (impurity, 15.3%), 125 (M⁺-CO, 6.3%), 111 (M⁺-CH₂CO, 18.9%, 55 (100%).

Preparation of 3-oxoethylidene-7-oxo-4-oxa-1-azabicyclo [3.2.0] heptane (V) from synthetic (I):—A solution of (I) (21.6 mg, 1 μmol) prepared by hydrogenation of benzyl 3-oxoethylidene-7-oxo-4-oxa-1-azabicyclo[3.2.0]heptane-2-c arboxylate (II) (90 mg, 0.31 mmol) in tetrahydrofuran (90 ml) was allowed to stand at room temperature for four hours when h.p.l.c. (conditions B) showed no further increase in the signal due to (E-V). The reaction mixture was evaporated to dryness and redissolved in ethyl acetete (50 ml). The ethyl acetate solution was washed with saturated NaHCO₃, 0.1M HCI and saturated NaCl, dried with magnesium sulphate, and evaporated to an oil (50 mg). Semi-preparative h.p.l.c. of this residue (conditions C) gave E-3-oxoethylidene-7-oxo-4-oxa-1-azabicyclo[3.2.0]heptane (8.3 mg, 49%) CD Δε_{237.4}6.1 CH₃CN); λₘₐₓ (CH₃CN) 249 nm (ε13,399 dm³ mol⁻¹ cm⁻¹); Vₘₐₓ 1 794, 1 669, 1 638, and 1 591 cm⁻¹; δ_{H} (250 MHz, CDCl₃) 3.19 (1H, d, J 16.7 Hz, 6β-H), 3.59 (1H, ddd, J 0.9, 2.8 and 16.6 Hz, 6α-H), 3.99 (1H, d, J17.5 Hz, 2α-H), 5.06 (1H, dd, J1.5 and 17.4 Hz 2β-H), 5.72 (1H, d, J 2.8 Hz, 5-H), 5.87 (1H, ddd, J 1.7, 1.7 and 3.5 Hz, 8-H) and 9.57 (1H, d, J 5.0 Hz, 9-H); addition of R-2,2,2-trifluoro-1-(9-anthryl)ethanol (1000%) caused separation of the signals due to the 2β proton chemical shift caused both sets of 2β protons to loose their smaller coupling. The two sets of 2β proton signals were in the ratio 2:1 m\z 153 (M⁺, 23%), 125 (M⁺-CO, 12%), 111 (M⁺CH₂CO, 24%) and 55 (M⁺-98, 100%). Further elution gave Z-3-oxoethylidene-7-oxo-4-oxa-1-azabicyclo[3.2.0]heptane containing 5% of the E isomer (2.3 mg, 14%), Vₘₐₓ 1 792 and 1 660 cm⁻¹ (br); dH (250 MHz, CDCl₃) 3.25 (1H, d, J 16.8, 6b-H), 3.52 (1H, ddd, J 0.8, 2.8 and 16.9 Hz, 6α-H), 3.85 (1H, d, J 16.8 Hz, 2α-H), 4.68 (1H, dd, J 0.8 and 17.0 Hz, 2β-H), 5.23 (1H, dt, J 1.0 and 7.9 Hz, 8-H), and 9.96 (1H, d, J 7.8 Hz); m/z 153 (M⁺, 24%) 125 (M+CO, 11%) 111, (M⁺⁻CH₂CO, 29%), and 55 (M⁺-98, 100%).

β-lactamase inhibition by dcl I 111 supernatant:—The β-lactamase inhibitory activity of dcl I 111 supernatant was estimated by the chromogenic cephalosporin assay of Reading and Farmer. A solution of nitrocephin (250 mg/ml) and RTEM JT4 β-lactamase from E. coli (5×10⁻³ BCD) in 0.05 M sodium phosphate at pH 7.3 was incubated at 37° C. The optical absorption of this reaction mixture at 492 nm was read in a 1 cm cuvette every minute for nine minutes to obtain an initial rate β-lactamase activity. Standard reactions containing aliquots of a solution of clavulanic acid (final concentrations: 1.0 and 0.5 mg/ml) in phosphate buffer were assayed in the same manner. Test reactions contained 43 hour dcl I 111 supernatant at known final dilutions. The clavulanic acid stock solution and the dcl I 111 supernatant were allowed to stand at room temperature, and were assayed for their initial rate of β-lactamase activity every half hour (see FIG. 4).

### EXAMPLE 3

A One-Step Purification of Clavulanic Acid Dehydrogenase (CAD) from Streptomyces clavuligerus SC2

SC2 is a reisolate of ATCC 27064. SC2 cells were grown in the standard carbohydrate medium (Woroniecki, et al. U.S. Pat. No. 4,795,809), harvested by centrifugation and then sonicated in the following buffer:—

50 mM MOPS

10% v/v glycerol

1 mM dithiothreitol (DTT)

1 mM ethylene diamine tetraacetic acid (EDTA)

1 mM phenylmethylsulphonyl fluoride (PMSF)

1 mM benzamidine-HCl

10 μM L-trans-epoxysuccinyl-leucylamido (4-guanidino)butane

pH 7.5

The cocktail of protease inhibitors was found to be necessary after loss of activity was observed on initial purification attempts.

Cell debris was removed by centrifugation and then the sonicate was made up to 1% streptomycin sulphate and left at 4° C. for 30 minutes. After this time the nucleic acid precipitate was removed by centrifugation and the remaining supernatant loaded onto the column.

The resin used was Red-Sepharose (Pharmacia), a dye resin known to have particular affinity for NADPH-dependent dehydrogenases (Pharmacia booklet, Affinity Chromatography, Principles and Methods). A 10 ml column was equilibrated in the above buffer and then 10 ml of treated sonicate was loaded on over one hour. After extensive washing with buffer, the column was pre-eluted with 10 ml of 10 mM NADH (to remove non-NADPH-dependent enzymes). Once the A₂₈₀ reading of the eluate had returned to baseline levels after washing with buffer, the column was eluted with an 8 hour linear gradient of NADPH (0-10 mM) in buffer, at 10 ml/hr. Ten minute fractions were collected and assayed for CAD activity. The assay for CAD is based on the well-known ‘KAG’ assay for β-lactamase inhibitors.

3.1 Assay.

Klebsiella pneumoniae, NCTC 11228, is grown on DST agar containing 5 μg ml⁻¹ penicillin G, with assay samples being placed in 8 mm wells. The samples are prepared by incubating the following:

25 μl column fraction

40 μl buffer (as above)

10 μl NADPH (10 mM)

25 μl clavulanic acid aldehyde (1 mg/ml approx)

at 21° C. for 10 minutes, before stopping the reaction with 300 μl cold buffer and adding 90 μl to the plate wells. In order to eliminate false results (due to non-enzymic effects/presence of clavulanic acid, etc.) each sample is also assayed in the absence of NADPH. After overnight incubation at 37° C., the zones of inhibition (due to clavulanate production) are measured. A small zone is observed even in the absence of CAD, due to residual aldehyde which is inhibitually active in its own right or other β-lactamase inhibitor (e.g., clavulanic acid).

N.B. The aldehyde of clavulanic acid is very unstable (half-life about 30 minutes at 21° C.) and must be prepared fresh within one hour of anticipated use and kept on ice.

3.2 Characterization

Protein with CAD activity was found to elute at 1-2 mM NADPH. (NB. Absorbance readings at 280 nm are not useful indicators of protein elution due to the absorbance of NADPH at this wavelength, therefore assays are required.) An SDS-PAGE analysis of the active fraction showed three bands—a prominent band at 28 kD and a fainter doublet at 26 kD. The gel was Western blotted onto an Immobilon-P membrane (Millipore). N-terminal amino acid sequencing analysis showed that all three protein bands shared the same N-terminal sequence as follows:—

| | |
|---|---|
| PSALQGKVALITGASSGIGE | (SEQ ID NO:3) |
| deduced from gene (M) | |
| PSALQGKVALITGASSGIGE | (SEQ ID NO:3) |

### EXAMPLE 4

Identification of the CAD Gene

4.1 Preparation of an S. clavuligerus Mutant (Dcl I 111) Blocked in Clavulanic Acid Biosynthesis.

The S. clavuligerus mutant Dcl I 111 was isolated during random screening procedures for high titre clavulanic acid producing mutants. The mutant accumulates clavulanic acid aldehyde.

4.2 Complementation of the lesion in Dcl I 111 by clones pBROC42 and pBROC41 and PWOR14

a. Preparation of pBROC41

Vector pBROC41 was constructed by ligating the BamHI/EcoRI fragment F-II in FIG. 2 from DNA fragment F-I in FIG. 2 (derivation described in EP 0 349 121 the entirety of which is disclosed in U.S. Pat. Nos. 5,759,831 and 6,037,156 both of which are hereby incorporated by reference in their entirety) into pIJ913 described by Hopwood et al (1985) Genetic Manipulation of Streptomyces , A Laboratory Manual. The John Innes Foundation.

b Preparation of pBROC42

Vector pBROC 42 was constructed by ligating a 2.5 kb BgIII fragment (F-III in FIG. 2) from DNA fragment F-I (FIG. 2) (for derivation see EP 0 349 121 the entirety of which is disclosed in U.S. Pat. Nos. 5,759,831 and 6,037,156) into pU702 described by Katz, E., Thompson, C. J., Hopwood, D. A. (1983) Journal General Microbiology 129, 2703-2714.

c Preparation of pWOR14

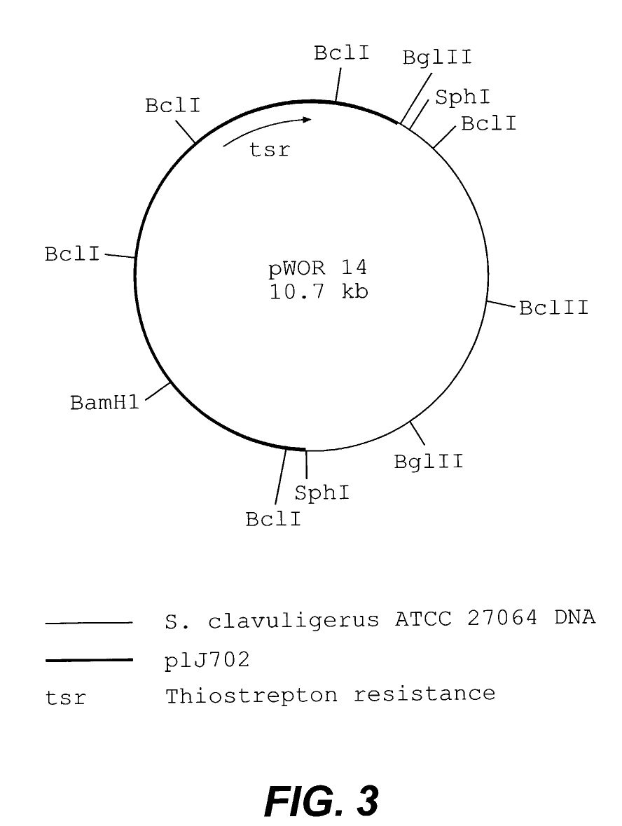

Vector pWOR14 (see FIG. 3) was constructed by ligating the 4.9 kb Sphl fragment F-IV in FIG. 2 from DNA fragment F-I in FIG. 2 (for derivation see EP 0 349 121 the entirety of which is disclosed in U.S. Pat. Nos. 5,759,831 and 6,037,156) into pIJ702 described by Katz, E., Thompson, C. J., Hopwood, D. A. (1983) Journal General Microbiology 129, 2703-2714.

d Complementation of mutant dclI 111

Vectors pBROC 41, pBROC 42 and pWOR14 were used to transform the dclI 111 as described in Bailey, C. R. et al Biotechnology 2, 808-811 (1984) and thiostrepton resistant transformants were streaked onto M5D plus thiostrepton at % ug/ml (EP 0349 121 the entirety of which is disclosed in U.S. Pat. Nos. 5,759,831 and 6,037,156).

Transformants of dclI 111 with pBROC41, pBROC42 and pWOR14 were tested for the repair of clavulanic acid by means of a plate bioassay. Cells of each transformant were stabbed onto M5D and incubated at 26 C for 6 days. The bioassay plates were then overpoured with soft blood agar (Oxoid) containing a strain of Klebsiella aerogenes described in Reading, C. and Cole, M. (1977) Antimicrob. Agents Chemorther. 11, 852-7, 0.02% terazolium salts and 5 ug/ml penicillin G. After overnight incubation at 26 C zones of inhibition had formed around the transformed colonies which were greater than those around the untransformed control (essentially zero).

Transformants containing pBROC41 and pBROC42 giving zones showing greater than the control culture were transferred to shake flask culture for accurate titre assessment. Cells from each colony were inoculated into 20 ml of seed medium (EP 0 349 121 the entirety of which is disclosed in U.S. Pat. Nos. 5,759,831 and 6,037,156) with the addition of thiostrepton (5 ug/ml final concentration) and grown for 3 days at 26° C. with shaking. 1 ml of the seed culture was then inoculated into a final stage medium (see EP 349 121 the entirety of which is disclosed in U.S. Pat. Nos. 5,759,831 and 6,037,156 both of which are hereby incorporated by reference in their entirety) containing 5 ug/ml thiostrepton and grown at 26° C. for up to four days. Samples of the final stage broth were withdrawn after three or four days growth and assayed for clavulanic acid productivity as described in Bird, A. E. et al. (1982) Analyst 1071241-1245 and Foulstone, M. and Reading, C. Antimicrob. Agents Chemother.22:753-762.

Results:—Clavulanic acid was produced in pBROC42 transformants of dclI 111 to 50% of the producing grandparent, and pBROC41 transformants to 15% of the producing grandparent. The grandparent strain is the producing parent of dclI 11. It was concluded that the DNA inserts II and III contain the gene for which there is a mutation in dcl I 111.

Conclusion :—CAD lies wholly within pBROC 41. It was concluded that pBROC41, pBROC42 and pWOR14 contain inserts complementing the dclI 111 mutation in the CAD gene.

4.3. Sequencing of clone pMG42

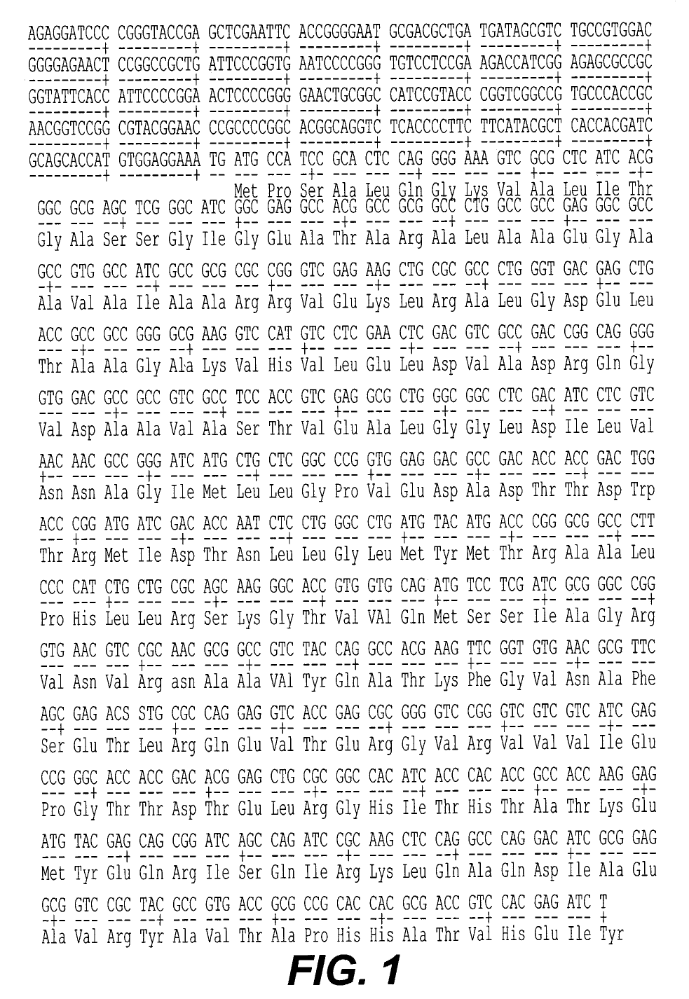

FIG. 2 shows a restriction map of part of the clavulanic acid gene cluster. Fragment F-III (/dcl I region) was sequenced from the EcoR1 site to the Bgl II site and an open reading frame found (FIG. 1). This (incomplete) sequence shows homology to known NADPH-dependent dehydrogenases from both prokaryotes and eukaryotes, such as rat corticoid 11β-dehydrogenase (Agaral et al., (1989) The Journal of Biological Chemistry 264:18939-18943 and ribitol dehydrogenase of Klebsiella aerogenes (Dothie et al., (1985) Biochemical Journal 230:569-578.

The sequence is incomplete and from transcriptional data a 1.2 kb mRNA has been assigned the CAD open-reading frame, i.e., the DNA sequence is approximately 80 bp short.

4.4 Identification of CAD Gene

The gene was identified by matching the sequence of the N-terminus of CAD obtained from S. clavuligerus to the sequence predicted from the open reading frame in a portion of S. clavuligerus chromosomal DNA which had been cloned on a vector (see 4.2 above).

The incomplete gene sequence gives a predicted protein size of at least 32 kD. This may reflect post-translational processing of a larger precursor to give the observed protein. Alternatively, and more probably, the unsequenced portion of the gene may be small, and thus the discrepancy between genetic and biochemical estimates of protein mass may be an artifact of the SDS-PAGE conditions.

### CONCLUSION

The results show that CAD is a 28 kD protein (by SDS-PAGE) and is encoded by the dehydrogenase-like open reading frame contained wholly within pBROC41 and pWOR14.

1. An isolated DNA fragment encoding a protein having clavulanic acid dehydrogenase (CAD) activity which hybridizes with an isolated 4.9 kb DNA from S. clavuligerus encoding a protein with CAD activity and having the endonuclease restriction pattern shown in fragment F-(IV) in FIG. 2 wherein the protein encoding sequence in the region bounded by EcoRI and BglII restriction sites is as shown in SEQ ID NO: 2, with the proviso that the isolated DNA fragment is not: (a) the 13.1 kb BglII to SphI fragment of S. clavuligerus; (b) the 10.8 kb EcoRI to BamHI fragment of S. clavuligerus; (c) the 8.8 kb BclI to BclI fragment of S. clavuligerus; (d) the 2.5 kb BglII to BglII fragment of S. clavuligerus; (e) the 60 kb BglII to BamHI fragment of S. clavuligerus; or (f) the 1 8.0 kb BclI to SphI fragment of S. clavuligerus.

2. A microorganism comprising the DNA of claim 1.

3. A vector comprising the DNA of claim 1.

4. A microorganism transformed with the vector of claim 3.

5. A microorganism having multiple copies of the DNA of claim 1.

6. A process to express the DNA fragment encoding a protein having CAD activity comprising culturing the microorganism of claim 2 under conditions sufficient for the production of said protein and recovering the protein from the culture medium.

7. An isolated DNA fragment encoding a protein with clavulanic acid dehydrogenase (CAD) activity wherein the DNA fragment is obtainable from S. clavuligerus and comprises the sequence in SEQ ID NO: 2, with the proviso that the isolated DNA fragment is not: (a) the 130.1 kb BglII to SphI fragment of S. clavuligerus; (b) the 10.8 kb EcoRI to BamHI fragment of S. clavuligerus; (c) the 8.8 kb BclI to BclI fragment of S. clavuligerus;
(d) the 2.5 kb BglII fragment of S. clavuligerus; (e) the 60 kb BglII to BamHI fragment of S. clavuligerus; or (f) the 18.0 kb BclI to SphI fragment of S. clavuligerus.

8. A microorganism comprising the DNA of claim 7.

9. A vector comprising the DNA of claim 7.

10. A microorganism transformed with the vector of claim 9.

11. A microorganism having multiple copies of the DNA of claim 7.

12. A process to express the DNA fragment encoding a protein having CAD activity comprising culturing the microorganism of claim 8 under conditions sufficient for the production of said protein and recovering the protein from the culture medium.

13. An isolated DNA fragment encoding a protein with CAD activity wherein the protein is clavulanic acid dehydrogenase (CAD) from S. clavuligerus having an apparent molecular mass of 28 kD as determined by SDS PAGE and comprising the sequence of amino acids beginning at the N-terminus as shown in SEQ ID NO:1, with the proviso that the isolated DNA fragment is not: (a) the 130.1 kb BglII to SphI fragment of S. clavuligerus; (b) the 10.8 kb EcoRI to BamHI fragment of S. clavuligerus; (c) the 8.8 kb BclI to BclI fragment of S. clavuligerus; (d) the 2.5 kb BglII to BglII fragment of S. clavuligerus; (e) the 60 kb BglII to BamHI fragment of S. clavuligerus; or (f) the 18.0 BclI to SphI fragment of S. clavuligerus.

14. A microorganism comprising the DNA of claim 13.

15. A vector comprising the DNA of claim 13.

16. A microorganism transformed with the vector of claim 15.

17. A microorganism having a high copy number of the DNA of claim 13.

18. A process to express the DNA fragment encoding a protein having CAD activity comprising culturing the microorganism of claim 14 under conditions sufficient for the production of said protein and recovering the protein from the culture medium.

19. An isolated DNA fragment encoding a protein having clavulanic acid dehydrogenase (CAD) activity that is at least 80% identical to SEQ ID NO:2.
